(19) United States
(12) Patent Application Publication
(10) Pub. No.: US 2007/0219610 A1
(43) Pub. Date: Sep. 20, 2007

(54) Stent with flap

(76) Inventor: Henry M. Israel, Bnei Brak (IL)

Correspondence Address: DEKEL PATENT LTD., DAVID KLEIN, BEIT HAROF'IM, 18 MENUHA VENAHALA STREET, ROOM 27, REHOVOT, 76209, IL

(21) Appl. No.: 11/378,308

(22) Filed: Mar. 20, 2006

(51) Int. Cl. A61F 2/06

(52) U.S. Cl. 623/1.11; 623/1.15

(57) ABSTRACT

An article including a stent including a flap that extends from a tubular support structure, the flap having an arcuate apex.

### FIELD OF THE INVENTION

This invention relates generally to implantable medical devices, and particularly to stents that can be used to block aneurysms.

### BACKGROUND OF THE INVENTION

An aneurysm is generally a localized blood-filled dilation of a vessel. One method of treating an aneurysm is to place a porous stent in the vessel at the aneurysm site. A porous stent can close an aneurysm over a short period of time, such as a week.

US Patent Application 20060030929 to Musbach describes a stent that includes an expandable framework and a plurality of rectangular flaps. Adjacent flaps overlap one another to form a wall region of predetermined shape. The stent is delivered to an aneurysm site and positioned such that the wall region blocks fluid flow into the aneurysm.

### SUMMARY OF THE INVENTION

The present invention seeks to provide a stent with a flap, which may be used to close or cover the ostium in a bifurcation, or block an aneurysm whether in a bifurcation or not, as is described in detail further hereinbelow.

There is provided in accordance with an embodiment of the present invention an article including a stent including a flap that extends from a tubular support structure, the flap having an arcuate apex. The arcuate apex may adjoin the tubular support structure. For example, the tubular support structure may have ends with a generally circular edge, and the arcuate apex may extend from one of the circular edges.

In accordance with an embodiment of the present invention the flap and the tubular support structure may have outer surfaces flush with each other. The flap and the tubular structure may have slots to provide flexibility during insertion. The flap may be coated with a polymer such as hydrogel that expands on exposure to a liquid thus providing the capability of closing the slots after insertion. The flap and the tubular support structure may be coated with a polymer such as to carry medication(s) to prevent restenosis in part and promote coagulation in a different part.

The flap may be gold coated and the tubular support structure may have gold bands to provide enhanced visibility under x-ray observation during insertion.

In accordance with an embodiment of the present invention the flap and the tubular support structure may be arranged so as to cover the ostium of a simple bifurcation. The flap may be arranged to curve into the bifurcation and the tubular structure may be open at one end to cover the walls of the ostium while the tubular support extends past the bifurcation to provide an anchor and support.

In accordance with an embodiment of the present invention the flap may have another arcuate apex that adjoins another tubular support structure. The flap may be intermediate ends of the tubular support structures.

In accordance with an embodiment of the present invention, two or more such stents may be arranged with each other such that their flaps overlap each other.

In accordance with an embodiment of the present invention, the flap may curve radially outwards away from a longitudinal axis of the tubular support structure.

In accordance with an embodiment of the present invention the arcuate apex may extend from a side wall of the tubular support structure.

There is also provided in accordance with an embodiment of the present invention, a catheter including a stent mounting portion, and a rotator operatively coupled to the stent mounting portion with a coupling, the rotator being operative to rotate the stent with the stent mounting portion.

There is also provided in accordance with an embodiment of the present invention a method including introducing a guidewire into a main lumen of a bifurcation, the guidewire including an outer slender tube with at least one deformable portion and an actuator disposed through a length of the outer slender tube and connected to the at least one deformable portion, introducing a catheter, with a stent deployed therewith, over the guidewire, the stent including a flap that extends from a tubular support structure, the flap having an arcuate apex, using the actuator to expand the at least one deformable portion outwards and thereby anchor the catheter in place, and expanding the stent, wherein the flap goes around an ostium of the bifurcation into a branch lumen of the bifurcation. The method may further include rotating the stent, and/or rotating and orienting the flap to block an aneurysm. A branch stent may be introduced from the catheter into the branch lumen.

### BRIEF DESCRIPTION OF THE DRAWINGS

The present invention will be understood and appreciated more fully from the following detailed description taken in conjunction with the drawings in which:

FIG. 1 is a simplified illustration of a stent with a tubular support structure and an end flap, constructed and operative in accordance with an embodiment of the present invention;

FIG. 2 is a simplified illustration of a stent with two tubular support structures and an intermediate flap extending therebetween, constructed and operative in accordance with an embodiment of the present invention;

FIG. 3 is a simplified illustration of two of the stents of FIG. 1 with the flaps overlapping each other, in accordance with an embodiment of the present invention;

FIG. 4 is a simplified illustration of a stent with a tubular support structure and an end flap that curves outwards, constructed and operative in accordance with an embodiment of the present invention;

FIG. 5 is a simplified illustration of two of the stents of FIG. 4 with the flaps overlapping each other, in accordance with an embodiment of the present invention;

FIG. 6 is a simplified illustration of a stent with a tubular support structure and a side flap, constructed and operative in accordance with an embodiment of the present invention;

FIGS. 7A, 7B and 7C are simplified sectional illustrations of a catheter useful in delivering the stents of the invention, in accordance with an embodiment of the present invention; and

FIGS. 8A, 8B, 8C, 8D and 8E are simplified illustrations of implanting a stent of the present invention at a bifurcated site, in accordance with an embodiment of the present invention.

### DETAILED DESCRIPTION OF EMBODIMENTS

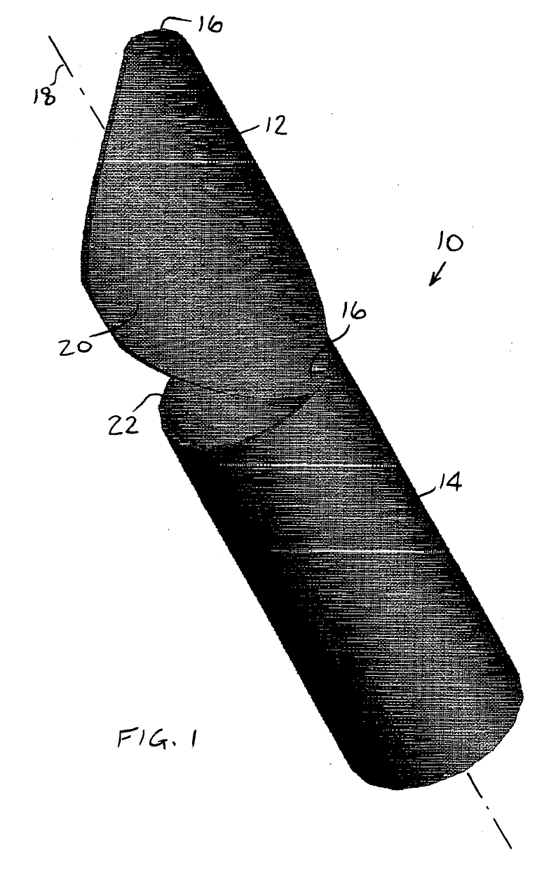

Reference is now made to FIG. 1, which illustrates a stent 10, constructed and operative in accordance with an embodiment of the present invention.

Stent 10 may include a flap 12 that extends from a tubular support structure 14. The stent 10 may be self-expanding, constructed of NITINOL, for example, or it may be balloon-expandable, constructed of stainless steel, for example. The stent 10 may be constructed of a suitable mesh or braided design, for example. Stent 10 may be coated, as least partially or full. For example, flap 12 and/or tubular support structure 14 may be coated with a polymer that expands when in contact with a liquid such as Hydrogel. As another example, flap 12 and/or tubular support structure 14 may be coated with a drug-eluting material, such as a polymer coating that emits a coagulation enhancing drug. As another example, flap 12 and/or tubular support structure 14 may be coated with an anti-restenosis drug.

Flap 12 may have a perimeter that is at least partially arcuate, and may have, for example, an arcuate apex 16 at one or both ends thereof. Tubular support structure 14 may be generally cylindrical in shape with a circular cross-section, although it may have other shapes and cross-sectional shapes, such as but not limited to, elliptic. Flap 12 may be curved about a longitudinal axis 18 so as to define a convex outer surface 20. The outer surface of flap 12 may be flush with the outer surface of tubular support structure 14. The radius of curvature of the convex outer surface of flap 12 may be equal to or different than the radius of the cross-section of tubular support structure 14.

In the non-limiting embodiment of FIG. 1, arcuate apex 16 adjoins tubular support structure 14. More specifically, in the embodiment of FIG. 1, tubular support structure 14 has ends 22 with a generally circular edge, and one of the arcuate apices 16 extends from one of the circular edges.

Flap 12 and/or tubular support structure 14 may have slots 23 formed therein to provide flexibility during insertion. Flap 12 may be coated with a coating 21, e.g., a polymer such as hydrogel that expands on exposure to a liquid thus providing the capability of closing the slots after insertion. The coating 21 may be a polymer that carries medication(s) to prevent restenosis in part and promote coagulation in a different part.

The coating 21 may be a gold coating. For example, flap 12 may be gold coated and the tubular support structure 14 may have gold bands to provide enhanced visibility under x-ray observation during insertion.

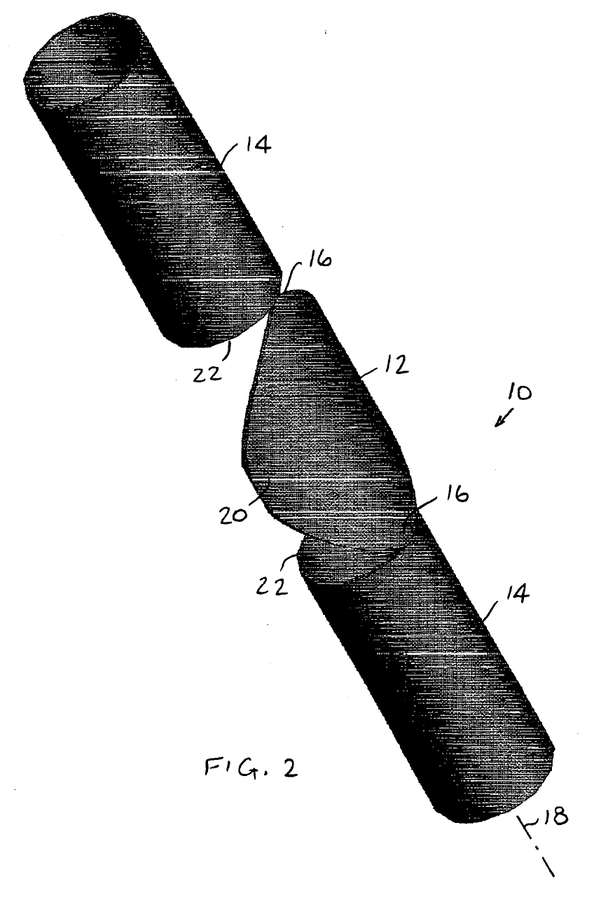

Reference is now made to FIG. 2, which illustrates a variation of stent 10, constructed and operative in accordance with another embodiment of the present invention. In this non-limiting embodiment, the other arcuate apex 16 of flap 12 adjoins another tubular support structure 14, such that flap 12 is intermediate ends 22 of the tubular support structures 14.

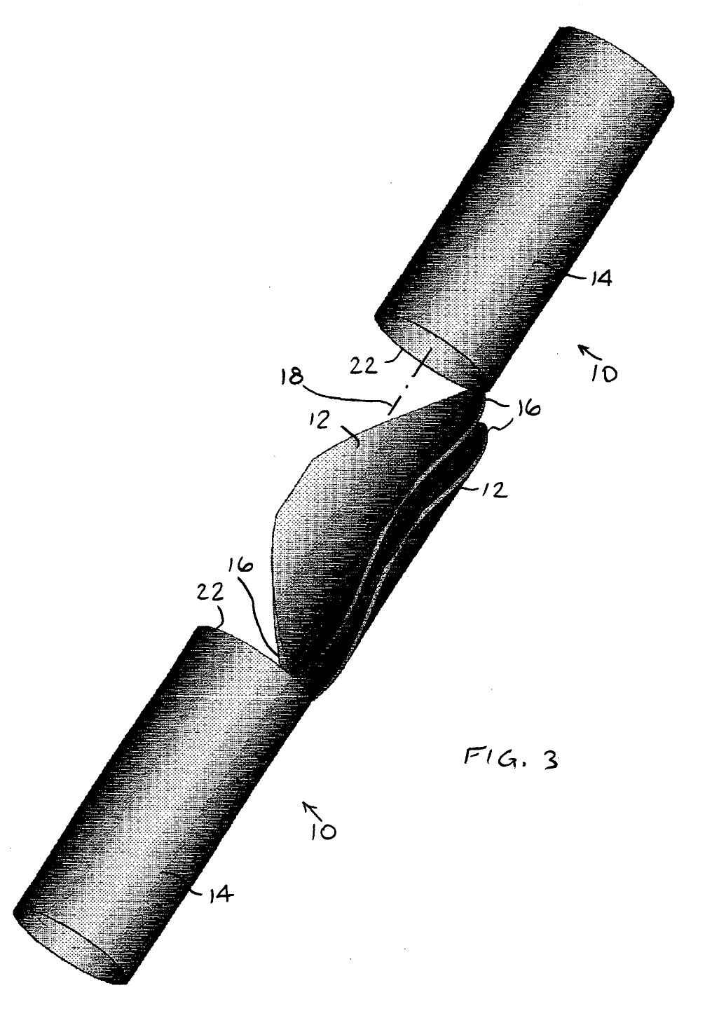

Reference is now made to FIG. 3. In this embodiment, two stents 10 are arranged with respect to each other such that their flaps 12 overlap each other.

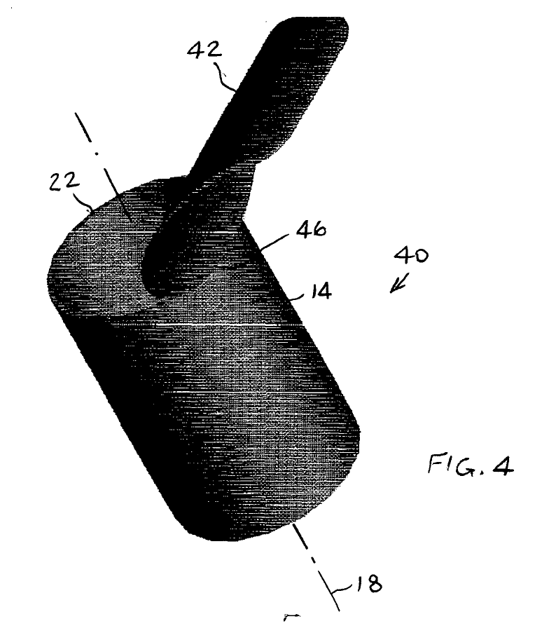

Reference is now made to FIG. 4, which illustrates a variation of stent 10, called stent 40, constructed and operative in accordance with another embodiment of the present invention. In this non-limiting embodiment, stent 40 has the same or similar tubular support structure 14 and an end flap 42 that curves radially outwards away from longitudinal axis 18 of tubular support structure 14. Similar to flap 12, the flap 42 may have a perimeter that is at least partially arcuate, and may have, for example, an arcuate apex 46 at one or both ends thereof. The arcuate apex 46 may adjoin tubular support structure 14 at an end 22 thereof.

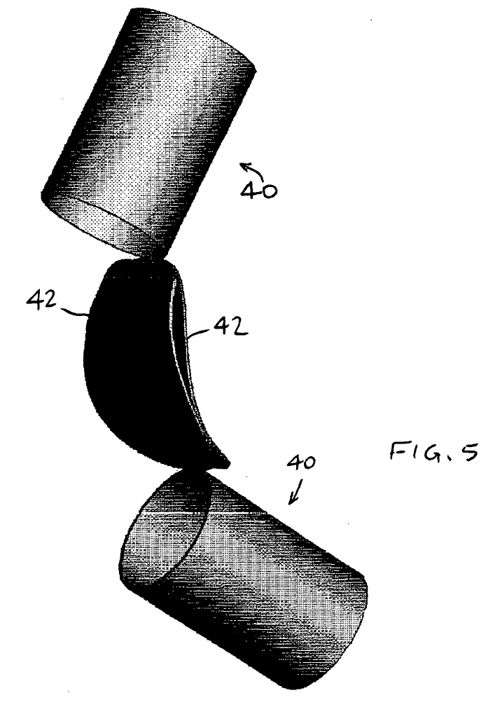

Reference is now made to FIG. 5. In this embodiment, two stents 40 are arranged with respect to each other such that their flaps 42 overlap each other.

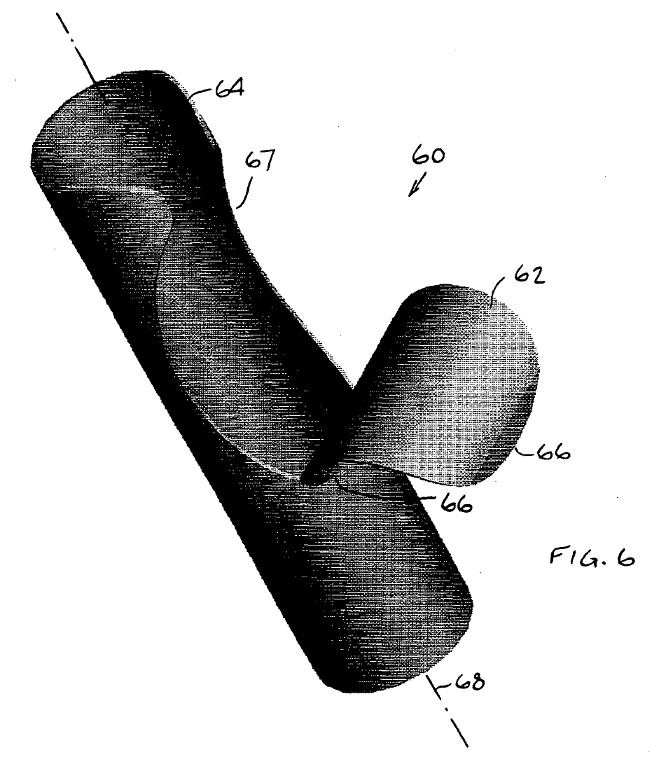

Reference is now made to FIG. 6, which illustrates a stent 60, constructed and operative in accordance with another embodiment of the present invention. Stent 60 fills the ostium of a bifurcation, providing a connection between stents in the branches and may include a flap 62 that extends from a tubular support structure 64 (64 is correct) to cover the junction of the two branches. As with stent 10, stent 60 may be self-expanding, constructed of NITINOL, for example, or it may be balloon-expandable, constructed of stainless steel, for example. The stent 60 may be constructed of a suitable mesh design, for example.

Flap 62 may have a perimeter that is at least partially arcuate, and may have, for example, an arcuate apex 66 at one or both ends thereof. Tubular support structure 64 may be generally cylindrical in shape with a circular cross-section, although it may have other shapes and cross-sectional shapes, such as but not limited to, elliptic. Tubular support structure 64 may have an arcuate side cutout 67 corresponding to the arcuate perimeter of flap 62. (Flap 62 may or may not be formed by cutting out cutout 67 and bending flap 62 outwards.) Flap 62 may curve radially outwards away from a longitudinal axis 68 of tubular support structure 64. In the non-limiting embodiment of FIG. 6, arcuate apex 66 adjoins tubular support structure 64 at an apex of side cutout 67, such that arcuate apex 66 extends from a side wall of the tubular support structure 64 the combination of support sections 64 and 67 provide covering and support for the ostium of the bifurcation.

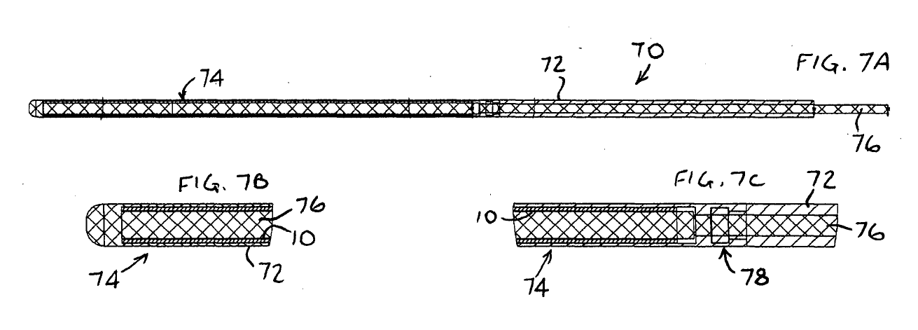

Reference is now made to FIGS. 7A-7C, which illustrate a catheter 70, useful in delivering any of the abovementioned stents of the invention, in accordance with an embodiment of the present invention.

Catheter 70 may include an outer sheath 72 with a stent mounting portion 74 at a distal end thereof for mounting therein a stent, such as stent 10 or any of the other stents of the invention. A rotator 76 may be disposed through the length of sheath 72. Rotator 76 may be a flexible rod that is operatively coupled to stent mounting portion 74 with a coupling 78. Rotation of rotator 76 causes rotation of the stent mounting portion 74, and with it, rotation of the stent mounted therein.

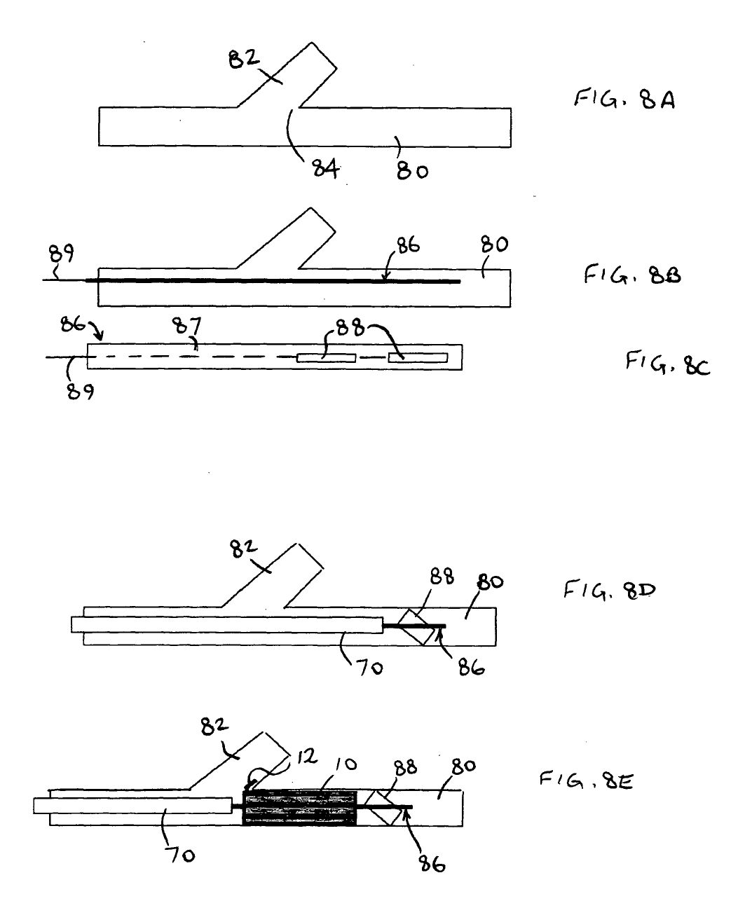

Reference is now made to FIGS. 8A-8E, which are simplified illustrations of implanting any of the stents of the present invention at a bifurcated site, in accordance with an embodiment of the present invention.

In FIG. 8A, a main lumen 80 (e.g., blood vessel in the brain) is shown with a bifurcation comprising a branch lumen 82 that branches from the main lumen 80 at an osmium 84 (opening of the branch lumen 82).

Reference is now made to FIGS. 8B and 8C. A guidewire 86 may be introduced into main lumen 80 (FIG. 8B). As seen in FIG. 8C, guidewire 86 may include an outer slender tube 87 with one or more deformable portions 88. An actuator, e.g., a slender pull wire 89, may be disposed through the length of outer slender tube 87 and connected to deformable portion(s) 88. Outer slender tube 87 and its deformable portion(s) 88 may be made of a durable material, such as but not limited to, a plastic, a shape memory alloy (such as a nickel-titanium alloy, e.g., NITINOL), or stainless steel which may be coated with a material such as polytetrafluoroethylene (PTFE). Guidewire 86 may have an outer diameter of about 0.38 mm, but the invention is not limited to this dimension.

Deformable portion(s) 88 may be in a collapsed orientation for placement in the body lumen. The slender pull wire 89 may serve as an actuator to effect movement of the deformable portion(s) 88 between a collapsed (or contracted, the terms being used interchangeably throughout) orientation and an expanded orientation.

Referring now to FIG. 8D, a stent delivery catheter, such as catheter 70, may be introduced over guidewire 86 and the distal most deformable portion 88 may be expanded outwards by the push or pull action of pull wire 89 that deforms portion 88 outwards. This anchors catheter 70 in place.

Referring now to FIG. 8E, in the case of a self-expanding stent, stent 10 may be released and self-expand, or in the case of a balloon-expandable stent, fluid may be introduced to expand the stent 10 (or other stent of the invention). Flap 12 goes around and reinforces the ostium 84 into branch lumen 82. Rotator 76 (FIGS. 7A-7C) may be used to rotate and orientate flap 12 and stent 10 as desired. In this manner, flap 12 may be directed to block an aneurysm, for example. A branch stent may now be introduced and expanded from catheter 70 into branch lumen 82.

It is appreciated that various features of the invention which are, for clarity, described in the contexts of separate embodiments, may also be provided in combination in a single embodiment. Conversely, various features of the invention which are, for brevity, described in the context of a single embodiment, may also be provided separately or in any suitable subcombination.

What is claimed is:

1. An article comprising:
a stent comprising a flap that extends from a tubular support structure, said flap having an arcuate apex.

2. The article according to claim 1, wherein said arcuate apex adjoins said tubular support structure.

3. The article according to claim 1, wherein said tubular support structure has ends with a generally circular edge, and said arcuate apex extends from one of said circular edges.

4. The article according to claim 1, wherein said flap and said tubular support structure have outer surfaces flush with each other.

5. The article according to claim 1, wherein said flap has another arcuate apex that adjoins another tubular support structure.

6. The article according to claim 1, further comprising a second stent that comprises a flap that extends from a tubular support structure, said flap having an arcuate apex, wherein the flap of said first-mentioned stent overlaps the flap of said second stent.

7. The article according to claim 1, wherein said flap curves radially outwards away from a longitudinal axis of said tubular support structure.

8. The article according to claim 1, wherein said arcuate apex extends from a side wall of said tubular support structure.

9. The article according to claim 7, wherein said arcuate apex extends from a side wall of said tubular support structure.

10. The article according to claim 1, wherein at least one of said flap and said tubular support structure have slots formed therein.

11. The article according to claim 1, wherein at least one of said flap and said tubular support structure are coated with a coating.

12. The article according to claim 11, wherein said coating comprises hydrogel that expands on exposure to a liquid.

13. The article according to claim 11, wherein said coating comprises a polymer that carries medication.

14. The article according to claim 11, wherein said coating comprises a gold coating.

15. An article comprising:
a catheter comprising a stent mounted in a stent mounting portion; and
a rotator operatively coupled to said stent mounting portion with a coupling, said rotator being operative to rotate said stent with said stent mounting portion.

16. The article according to claim 15, wherein at least a portion of said stent is coated with a coating.

17. A method comprising:
introducing a guidewire into a main lumen of a bifurcation, said guidewire comprising an outer slender tube with at least one deformable portion and an actuator disposed through a length of said outer slender tube and connected to said at least one deformable portion;
introducing a catheter, with a stent deployed therewith, over said guidewire, said stent comprising a flap that extends from a tubular support structure, said flap having an arcuate apex;
using said actuator to expand said at least one deformable portion outwards and thereby anchor said catheter in place; and
expanding said stent, wherein said flap goes around an ostium of said bifurcation into a branch lumen of said bifurcation.

18. The method according to claim 17, further comprising rotating said stent.

19. The method according to claim 17, further comprising rotating and orienting said flap to block an aneurysm.

20. The method according to claim 17, further comprising introducing a branch stent from said catheter into said branch lumen.
